(19) United States
(12) Patent Application Publication
(10) Pub. No.: US 2020/0138983 A1
(43) Pub. Date: May 7, 2020

(54) USING ENDOSCOPY AS A CHANNEL TO DELIVER CANCER-OR TISSUE-TARGETED THERANOSTIC AGENTS

(71) Applicant: ASTRID PHARMA CORP., Davis, CA (US)

(72) Inventors: R. Holland Cheng, Davis, CA (US); Chun-Chieh Chen, Davis, CA (US)

(21) Appl. No.: 16/541,909

(22) Filed: Aug. 15, 2019

Related U.S. Application Data
(60) Provisional application No. 62/719,084, filed on Aug. 16, 2018.

(51) Int. Cl. A61K 49/14; A61B 1/018; A61B 1/07; A61B 1/00; A61B 1/05; A61B 1/04; C07K 14/005; C12N 7/00; A61K 49/00; A61K 41/00

(52) U.S. Cl. CPC A61K 49/14; A61B 1/018; A61B 1/07; A61B 1/00195; A61B 1/00045; A61B 1/05; C12N 2770/28133; C07K 14/005; C12N 7/00; A61K 49/0056; A61K 41/0052; C12N 2770/28123; A61B 1/043

(57) ABSTRACT

The present disclosure provides a cancer-specific or tissue specific targeting theranostic capsule using hepatitis E viral nanoparticle (HEVNP) to enhance the accuracy of cancer diagnosis in endoscopic examinations, as well as treatment, for example hyperthermia treatment, after diagnosis. The present disclosure also provides a method of delivering a theranostic agent using the endoscopic apparatus, as well as a non-transitory computer readable medium storing a program that causes a computer to execute the method of the present invention.

### CROSS REFERENCE TO RELATED APPLICATIONS

The present application claims priority from U.S. provisional patent application 62/719,084 filed on Aug. 16, 2018, entitled “USING ENDOSCOPY AS A CHANNEL TO DELIVER CANCER- OR TISSUE-TARGETED THERANOSTIC AGENTS”, the content of which is hereby incorporated by reference in their entirety for all purposes.

### SEQUENCE LISTING

A sequence listing was filed in this case via EFS-Web as a PDF file under 37 CFR 1.821(1)(c), and a computer readable form (CRF) of the sequence listing is being filed as an ASCII text file via EFS-Web under 37 CFR 1.821(2)(b). The ASCI text file has a file name of “16541909SequenceListing,” a date of creation of “Jan. 16, 2020,” and has a file size of “41 KB.” The PDF file of the sequence listing is identical to the sequence listing filed in the ASCII text file, whereby this sequence listing is hereby incorporated by reference.

### BACKGROUND OF THE INVENTION

### Technical Field

The present invention relates to a cancer-specific or tissue targeting theranostic capsule, as well as an endoscopic apparatus and a method for delivering the same. In particular, the present invention relates to a cancer-specific or tissue-specific targeting theranostic capsule using hepatitis E viral nanoparticle (HEVNP), and an endoscopic apparatus and method which enable delivery of the cancer-specific or tissue targeting theranostic capsule using hepatitis E viral nanoparticle (HEVNP).

### Description of the Prior Art

Endoscopy is a simple procedure which allows a doctor to look inside the human body using an instrument called an endoscope. An endoscope generally consists of a rigid or flexible tube, a light delivery system to illuminate the organ or object under inspection, a lens system to transmit images from the objective lens to a viewer or camera and/or videoscope for image capture, a water channel to deliver water/liquid to the front end of the tube for medical purposes such as cleaning, and additional channels to allow entry of medical instruments or manipulators. A doctor may use endoscopy for any of the following:
investigation of digestive system problems and symptoms including nausea, vomiting, abdominal pain, difficulty swallowing, and gastrointestinal bleeding.
confirmation of a diagnosis, most commonly by performing a biopsy to check for conditions such as anemia, bleeding, inflammation, and cancers of the digestive system.
giving treatment, such as cauterization of a bleeding vessel, widening a narrow esophagus, removal of a polyp or foreign object.
   Endoscopy procedures primarily rely on captured visual images without the use of cancer/tissue targeted therapeutics or diagnostics.

Nanoparticles have been used for cancer/tissue targeting for diagnostic and therapeutic applications. Various theranostic agents have been used in diagnosing and/or treating various diseases. Specific applications of the theranostic agents include diagnosing and/or treating diseases such as cancer but not only limited in cancers. Recently, nanoparticles are increasingly used as delivery carriers of theranostic agents for targeted diagnosis and treatment of cancers and/or other diseases.

The typical way for most therapeutic delivery systems is via injection. Moreover, some may be delivered via oral delivery as well, such as HEVNP. However, both oral administration and injection have their own drawbacks. For oral administration of nanoparticles, one challenge is overcoming low bioavailability due to the harsh environment in GI tracts. On the other hand, injection via needles not only causes discomfort, and also brings an off-target side-effect since the drugs are delivered via circulation system.

In diagnosing and/or treating cancer, Hepatitis E viral nanoparticles (HEVNP), which is modified from Hepatitis E virus-like particles (HEV-VLPs) can serve as nano-carriers for targeted delivery of diagnostics, such as MRI or PET imaging enhancing reagents, and therapeutics regimes, such as DNA/RNA and a variety of chemotherapeutics. HEVNPs are composed of derived Hepatitis E viral capsid protein that can be produced by expression of HEV capsid protein Open Reading Frame 2 (ORF2) in a eukaryotic expression system, such as E. coli, insect cells, or yeast. HEVNPs are stable in acid and proteolytic environments, a feature that is required for the natural transmission route of HEV. Thus, HEVNPs represent a promising nano-carrier that can be exploited, e.g., for chemotherapeutic delivery, vaccination, gene delivery and/or diagnostic delivery via both circulation and mucosal delivery routes.

Hepatitis E virus (HEV) is known to cause severe acute liver failure. HEV belongs to the genus Hepevirus in the family Hepeviridae. HEV contains a single-stranded positive-sense RNA molecule of approximately 7.2-kb. The RNA is 3′ polyadenylated and includes three open reading frames (ORF). ORF1 encodes viral nonstructural proteins, located in the 5′ half of the genome. ORF2 encodes a protein-forming viral capsid, located at the 3′ terminus of the genome. ORF3 encodes a 13.5-kDa protein, overlapped with C-terminus of ORF1 and N-terminus of ORF2. ORF3 is associated with the membrane as well as with the cytoskeleton fraction. “Hepatitis E virus” or “HEV” refers to a virus, virus type, or virus class, which i) causes water-borne, infectious hepatitis; ii) is distinguished from hepatitis A virus (HAV), hepatitis B virus (HBV), hepatitis C virus (HCV), or hepatitis D virus (HDV) in terms of serological characteristics; and iii) contains a genomic region that is homologous to a 1.33 kb cDNA inserted in pTZKF1(ET1.1), a plasmid embodied in a E. coli strain deposited in American Type Culture Collection (ATCC) with accession number 67717.

The terms “capsid protein” and “modified capsid protein,” with reference to HEV, refer to a mature or modified (e.g., truncated, recombinantly mutated, or chemically derivatized) HEV open reading from 2 (ORF2) polypeptide. As used herein, reference to such ORF2 polypeptides or proteins is meant to include the full-length polypeptide, and fragments thereof, and also include any substitutions, deletions, or insertions or other modifications made to the ORF2 proteins. The capsid proteins must be capable of forming a virus like particle (VLP). Typically the capsid protein contains at least residues 112-608 of HEV ORF2, although the capsid protein can tolerate various additional substitutions, deletions, or insertions so long as they are tolerated without abrogating VLP formation.

The term “modified capsid protein” refers to a capsid protein, or portion thereof (i.e., less than full length of the capsid protein), in which modifications such as one or more of additions, deletions, substitutions are presented yet the resultant modified capsid protein remain capable of forming a VLP. These modifications include those described in U.S. Pat. Nos. 8,906,862 and 8,906,863, WO2015/179321. For instance, a heterologous polypeptide may be inserted into the capsid protein or a portion thereof, at locations such as within segment 483-490, 530-535, 554-561, 573-577, 582-593, or 601-603, or immediately after residue Y485, see U.S. Pat. Nos. 8,906,862 and 8,906,863. As an another example, WO2015/179321 describes further examples of modified capsid protein in which a surface variable loop of the P-domain of HEV ORF2 is modified to incorporate one or more cysteines or lysines that are not otherwise present in the wild-type capsid protein sequence. Alternatively, or additionally, the term “modified capsid protein” refers to a capsid protein, or portion thereof, in which the C-terminus (e.g., position 608) of HEV ORF2 is modified to incorporate one or more cysteines or lysines that are not otherwise present in the wild-type capsid protein sequence. Alternatively, or additionally, the term “modified capsid protein” refers to a capsid protein, or portion thereof, in which a cysteine or lysine (e.g., a cysteine or lysine of a surface variable loop of the P-domain of HEV ORF 2 or a cysteine/lysine recombinantly introduced at position 608) is chemically derivatized to covalently conjugate to the protein at least one heterologous atom or molecule. The cysteine or lysine can be inserted such that the HEV ORF2 protein length is increased, or the cysteine or lysine can replace one or more residues of a P-domain surface variable loop and/or C-terminus.

Generally, modified capsid proteins retain the ability to form HEV VLPs. In some cases, the one or more cysteines or lysines are conjugated to a bioactive agent (e.g., a cell-targeting ligand such as the peptide LXY30). P-domain surface variable loops include one or more of, e.g., residues 475-493; residues 502-535; residues 539-569; residues 572-579; and residues 581-595 of HEV ORF 2 (SEQ ID NO:1, 2, 3, 4, 5, or 6). P-domain surface variable loops further include the residues of polypeptides comprising an amino acid sequence that is at least about 80%, 85%, 90%, 95%, 99%, or more identical to one or more of SEQ ID NOS:1, 2, 3, 4, 5, or 6 and that correspond to one or more of residues 475-493; residues 502-535; residues 539-569; residues 572-579; and residues 581-595 of SEQ ID NOS:1, 2, 3, 4, 5, or 6.

The term “virus-like particle” (VLP) refers to an icosahedral shell (e.g., T1 or T3) formed by a capsid protein. VLPs are not infectious due to the lack of a viral genome. “VLP” refers to a nonreplicating icosahedral viral shell, derived from hepatitis E virus capsid protein HEV ORF2, a portion thereof. VLPs can form spontaneously upon recombinant expression of the protein in an appropriate expression system. In some embodiments, the VLP is formed from a modified capsid protein, e.g., a capsid protein containing one or more cysteine/lysine residues in a surface variable loop of HEV ORF2, or a portion thereof. An HEV VLP can contain a mixture of modified and/or unmodified HEV ORF2 proteins.

The term “acid and proteolytically stable” in the context of an HEV VLP refers to an HEV VLP that is resistant to the acid and proteolytic environments of a mammalian digestive system. Methods of assessing acid and proteolytic stability are described in Jariyapong et al., 2013, and include, but are not limited to subjecting an HEV VLP to an acid (e.g., pH of, or of about, 6, 5.5, 5, 4.5, 4, 3.5, 3, 2.5, or 2) and/or proteolytic environment (e.g., trypsin and/or pepsin) and examining the contacted HEV VLP by electron microscopy, gel filtration chromatography, or other suitable method to determine whether the quaternary structure (e.g., T=1, T=3, icosahedron, dodecahedron, etc.) of the HEV VLP is retained. A population of HEV VLPs (e.g., modified or unmodified) can be incubated under acid and/or proteolytic conditions for a suitable period of time (e.g., for at least, or for at least about, 1, 2, 3, 4, 5, 10, 15, 20, 30, 45, or 60 minutes) and then tested to determine the extent of quaternary structure retention. In this context, an acid and proteolytically stable modified HEV VLP refers to a modified HEV VLP that when incubated as a population of VLPs under acid and/or proteolytic conditions and assayed by electron microscopy, at least 10%, 25%, 50%, 75%, 90%, 95%, 99%, or 100% of the VLPs of the population retain their quaternary structure.

The term “heterologous” as used in the context of describing the relative location of two elements, refers to the two elements such as nucleic acids (e.g., promoter or protein encoding sequence) or proteins (e.g., an HEV ORF2 protein, or portion thereof, or modified capsid protein and another protein) that are not naturally found in the same relative positions. Thus, a “heterologous promoter” of a gene refers to a promoter that is not naturally operably linked to that gene. Similarly, a “heterologous polypeptide” or “heterologous nucleic acid” in the context of an HEV VLP or HEV capsid protein is one derived from a non-HEV origin.

The term “encapsulation,” or “encapsulated,” as used herein refers to the envelopment of a heterologous substance, such as a heterologous nucleic acid or protein, a chemotherapeutic, an imaging agent, a ferrite nanoparticle etc., within the VLPs defined herein.

A “label,” “detectable label,” or “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins that can be made detectable, e.g., by incorporating a radioactive component into the peptide or used to detect antibodies specifically reactive with the peptide. Typically a detectable label is a heterologous moiety attached to a probe or a molecule with defined binding characteristics (e.g., a polypeptide with a known binding specificity or a polynucleotide), so as to allow the presence of the probe/molecule (and therefore its binding target) to be readily detectable. The heterologous nature of the label ensures that it has an origin different from that of the probe or molecule that it labels, such that the probe/molecule attached with the detectable label does not constitute a naturally occurring composition.

The term “treat” or “treating,” as used in this application, describes to an act that leads to the elimination, reduction, alleviation, reversal, or prevention or delay of onset or recurrence of any symptom of a relevant condition. In other words, “treating” a condition encompasses both therapeutic and prophylactic intervention against the condition.

The term “effective amount” as used herein refers to an amount of a given substance that is sufficient in quantity to produce a desired effect. For example, an effective amount of HEV nanoparticle (HEVNP) encapsulating insulin is the amount of said HEVNP to achieve a detectable effect, such that the symptoms, severity, and/or recurrence chance of a target disease (e.g., diabetes) are reduced, reversed, eliminated, prevented, or delayed of the onset in a patient who has been given the HEVNP for therapeutic purposes. An amount adequate to accomplish this is defined as the “therapeutically effective dose.” The dosing range varies with the nature of the therapeutic agent being administered and other factors such as the route of administration and the severity of a patient's condition.

The term “patient” as used herein refers to a vertebrate animal, e.g., of avian or mammalian species, especially a mammal (for example, a bull/cow, pig, sheep/goat, horse, rabbit, rodent, dog, cat, fox, etc.) including a primate such as a chimpanzee, a monkey or a human.

### SUMMARY OF THE INVENTION

In view of the problems set forth above, the inventors provide a endoscopic apparatus and a method for delivering a theranostic agent, for example modified HEVNPs, with excellent targeting to avoid off-target side-effect. Endoscopy is used to semi-specifically deliver theranostic nanoparticles to potential cancer sites after optical imaging. The invention involves a tumor-targeting nanoparticle with tumor-targeting ligands that are conjugated on the HEVNP surface. These HEVNPs will also encapsulate magnetic nanoparticles, such as ferrite, that will function as alternating magnetic field (AMF) inducible heating reagents for hyperthermia-based treatment. Here, a protocol is disclosed as example of the invention to deliver the cancer targeted nanoparticles to colon cancer sites through colonoscopy. The multi-functional HEVNPs can be used as diagnostic agent through fluorescence detection or MRI detection, and/or as a therapeutic agent for hyperthermia treatment by applying AMF and or ultrasonic waves.

In one aspect of the present invention, an endoscopic apparatus is provided. The endoscopic apparatus comprises a tubular body having a front end and an operating end, and a controller coupled to the operating end of the tubular body. The tubular body comprises a fluid channel disposed inside the tubular body, the fluid channel having a fluid inlet at the operating end and a fluid outlet at the front end. A theranostic agent source is coupled to the fluid inlet of the fluid channel and configured to supply a theranostic agent through the fluid channel. The controller is configured to control various operations of the endoscopic apparatus. During a diagnosis and treatment process for a subject having a portion to be diagnosed and treated, the endoscopic apparatus is introduced into a body of subject, such that the front end of the tubular body is disposed at a predetermined distance from the portion to be diagnosed and treated.

In another aspect of the present invention, a method of delivering a theranostic agent using an endoscopic apparatus is provided. The endoscopic apparatus comprises a tubular body, an imaging system disposed inside the tubular body, an image displaying system coupled to the imaging system, an illumination system disposed inside the tubular body, a fluid channel disposed inside the tubular body, and a theranostic agent source coupled to the fluid channel The method comprises: preparing a subject having a portion to be diagnosed and treated; introducing the endoscopic apparatus into a body of the subject having a portion to be diagnosed and treated; illuminating an area around a front end of the endoscopic apparatus introduced into the body of the subject; delivering the theranostic agent from the theranostic agent source through the fluid channel to a portion to be diagnosed and treated; capturing, using the imaging system, images within the area around the front end of the endoscopic apparatus; displaying images captured by the imaging system with the image displaying system; and determining the condition of the theranostic agent supplied on the portion to be diagnosed and treated based on the images captured by the imaging system and displayed on the image displaying system.

In yet another aspect of the present invention, a non-transitory computer readable medium is provide. The non-transitory computer readable medium stores a program causing a computer to execute the method of the present invention.

In an aspect of the present invention, the present invention provides a composition delivered using the method of the present invention. The composition comprising (a) modified capsid protein that comprises at least a portion of hepatitis E virus (HEV) open Reading Frame 2 (ORF2) protein and is able to form an HEV virus like particle (VLP); and/or (b) cancer/tumor targeting ligands either in the form of peptides or small molecules chemically conjugated and/or genetic engineered onto the surface of HEV VLP formed by the modified capsid protein; and/or (c) fluorescence dye and/or X-ray detectable agent/nanoparticles and/or MRI detectable agent/nanoparticles either chemically conjugated and/or encapsulated within the HEV VLP formed by the modified capsid protein; and/or (d) heat-inducible agents/nanoparticles by ultrasonic wave and/or alternating magnetic field (AMF) and/or visible light either conjugated on the surface of HEV VLP and/or encapsulated within the HEV VLP formed by modified capsid protein; and/or anti-cancer therapeutics either in forms of peptides, nucleic acids, small molecules, inorganic particles either conjugated on the surface of HEV VLP and/or encapsulated within the HEV VLP formed by modified capsid protein. Typically, the modified ORF2 protein is less than full length of the wild-type protein (e.g., any one of those provided in SEQ ID NOs:1-6).

In some embodiments, the modified capsid protein is less than full length of HEV ORF2 protein; it comprises the M domain, segment 318-451, and/or the P domain, segment 452-606 of the HEV ORF 2 protein of SEQ ID NO:1, 2, 3, 4, 5, or 6; and it comprises a heterologous polypeptide sequence inserted into the portion of HEV ORF2 protein within segment 342-344, 402-409, 483-490, 530-535, 554-561, 573-577, 582-593, or 601-603 of SEQ ID NO:1, 2, 3, 4, 5, or 6. In some embodiments, the heterologous polypeptide sequence is inserted immediately after residue T342, E407, or Y485 of SEQ ID NO:1, 2, 3, 4, 5, or 6. In some embodiments, the heterologous polypeptide may be involved in targeting cancer cells for delivery of diagnostics and/or therapeutics, for example, the most widely used homing peptide, RGD (Arg-Gly-Asp) peptide or cyclic RGD peptide, which shows strong affinity for integrins vb 3 and vb 5, or homing peptides that specifically target HCC include TTPRDAY, FQHPSFI (HCBP1), SFSIIHTPILPL (SP94), RGWCRPLPKGEG (HCl), AGKGTPSLETTP (A54), KSLSRHDHIHHH (HCC79) and AWYPLPP.

In some embodiments, the modified capsid protein is able to form an acid and proteolytically stable HEV VLP and has at least one residue T342, E407, Y485, T489, S533, N573, or T586 of SEQ ID NO:1, 2, 3, 4, 5, or 6 substituted with a cysteine or lysine, and the cysteine or lysine is optionally chemically derivatized. In some embodiments, the cysteine or lysine is alkylated, acylated, arylated, succinylated, oxidized, or conjugated to a detectable label or cancer cell targeting ligand. For example, the detectable label may comprise a fluorophore, a superparamagnetic label, an MRI contrast agent, a positron emitting isotope, or a cluster of elements of group 3 through 18 having an atomic number greater than 20. In some embodiments, the detectable label comprises a gold nanocluster. In another example, the cancer cell targeting ligand is the heterologous polypeptide may be involved in targeting cancer cells for delivery of theranostic, for example, the most widely used homing peptide, RGD (Arg-Gly-Asp) or cyclic RGD peptide, or homing peptides that specifically target HCC include TTPRDAY, FQHPSFI (HCBP1), SFSIIHTPILPL (SP94), RGWCRPLPKGEG (HCl), AGKGTPSLETTP (AM), KSLSRHDHIHHH (HCC79) and AWYPLPP.

In some embodiments, the patient is an animal, especially a mammal such as a primate including a human.

### BRIEF DESCRIPTION OF THE DRAWINGS

FIG. 1 is a schematic view of an endoscopic apparatus according to an embodiment of the present invention.

FIG. 2 is a flow chart of an exemplary diagnosing method according to an embodiment of the present invention.

FIG. 3 is a flow chart of an exemplary treating method according to an embodiment of the present invention.

FIGS. 4A-4C illustrate structures of HEV-VLP presented as secondary structure for the M domain.

FIG. 5 illustrates the structure of M domain presented as a secondary structure.

FIG. 6 illustrates structures of HEV-VLP and HEV PORF2 dimer presented as a solid surface.

FIG. 7 illustrates similar VLP formation of all two HEV-Cys VLPs represented by electron micrographs of negative stained VLPs.

FIG. 8 illustrates surface exposure assay of Cys sites on P domains of HEV-573C, M domains of HEV-342C and HEV-407C.

### DETAILED DESCRIPTION OF THE PREFERRED EMBODIMENTS

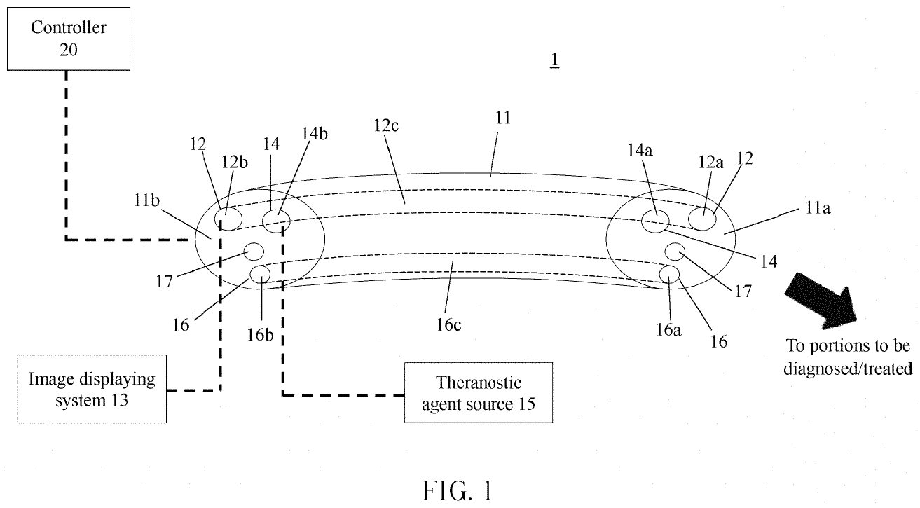

FIG. 1 is a schematic view of an endoscopic apparatus 1 according to an embodiment of the present invention. As shown in FIG. 1, the endoscopic apparatus 1 includes a tubular body 11, an imaging system 12, an image displaying system 13, a fluid channel 14, a theranostic agent source 15, and a illumination system 16. The imaging system 12, fluid channel 14 and illumination system 16 are provided inside the tubular body 11.

The tubular body 11 includes a front end 11a and an operating end 11b. When the endoscopic apparatus 1 is used in a targeted treatment process, the front end 11a can be inserted into a body of a subject, such that the front end 11a of the tubular body 11 is disposed at a predetermined distance from a portion to be diagnosed and/or treated inside the body of the subject. During the targeted treatment process, an operator may monitor and/or control the operation of the endoscopic apparatus 1 at the operating end 11b.

The imaging system 12 includes an image capturing end 12a at the front end 11a and an image receiving end 12b at the operating end 11b. In addition, the imaging system 12 includes an image transmitting device 12c provided between the image capturing end 12a and the image receiving end 12b. The image displaying system 13 is coupled to the image receiving end 12b of the imaging system 12, and displays the images transmitted from the image capturing end 12a via the image transmitting device 12c. From the images displayed on the image displaying system 13, the operator may determine whether the targeted diagnosis/treatment process is performed in a desired manner In one embodiment, the image displaying system 13 may be an eyepiece. In another embodiment, the image displaying system 13 maybe a camera.

In some embodiments, the tubular body 11 has a flexible structure. The image transmitting device 12c may comprise an optical fiber device, which can be configured to pass through/crossover a channel of the tubular body. In some embodiments, the tubular body 11 comprises a rigid structure. The image transmitting device 12c may comprise a relay lens system.

The fluid channel 14 includes a fluid inlet 14b at the operating end 11b and a fluid outlet 14a at the front end 11a. The theranostic agent source 15 is coupled to the fluid inlet 14b of the fluid channel 14 and supplies theranostic agent which enters the fluid inlet 14b, through the fluid channel 14, and exits the fluid outlet 14a to the portion to be diagnosed and/or treated inside the body of the subject. The theranostic agent can be delivered continuously or sprayed in a pressurized manner by a pump connected to the fluid channel 14.

One example of the theranostic agent is nanoparticle suspension including tumor-targeting nanoparticles with tumor-targeting ligands, such as RGD derived motifs, that are conjugated on the surfaces of the nanoparticles. In some embodiments, the nanoparticles comprise encapsulate magnetic nanoparticles, for example but not limited to, ferrite that functions as alternating magnetic field (AMF) inducible heating reagents for hyperthermia-based treatment. In another example, HEVNPs with both cancer targeting ligands and fluorescence dyes conjugated on the surface and the magnetic particles, such as ferrite derived nanoparticles, encapsulated in the interior, can be used as diagnostic agents through fluorescence detection or MRI detection, and/or as a therapeutic agent for hyperthermia treatment induced by AMF.

The illumination system 16 includes a light inlet end 16b at the operating end 11b and a light outlet end 16a at the front end 11a. A light source is coupled to the light inlet end 16b. The illumination system 16 further includes a light transmitting device 16c. During the operation of the endoscopic apparatus 1, the light emitted by the light source enters the light inlet end 16b, travels through the light transmitting device 16c, and exits the light outlet end 16a. In such manner, a region around the front end 11a can be illuminated, so that the imaging system 12 can capture sufficiently clear images for the operator to determine the condition of the portion to be diagnosed and/or treated.

The endoscopic apparatus further comprises a controller 20 coupled to the operating end 11b of the tubular body 11 through a communication interface (not shown). The controller 20 includes a computer-readable media which stores instructions for controlling various operations of the endoscopic apparatus 1. In an embodiment, the computer readable media included in the controller 20 stores a program which includes instructions for executing the method of present disclosure, as will be further described below. In some embodiments, the controller 20 is, for example, servers, desktops, laptops, consumer devices or appliances such as mobile phones, tablets, television sets, or any other processor-based devices, or combinations thereof.

In some embodiments, the endoscopic apparatus includes an optional instrument channel 17, as shown in FIG. 1. When treating a portion to be treated inside the body of the subject, a surgery tool is inserted through the instrument channel 17 and reach the portion to be treated for performing a surgery to the portion to be treated.

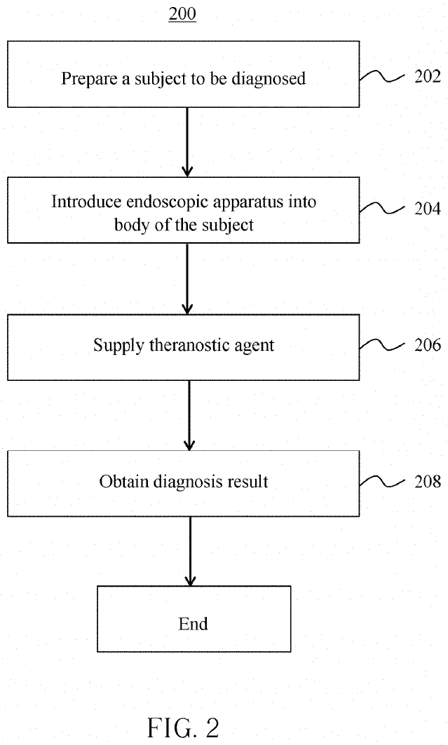

The present disclosure provides methods of using endoscopy as a channel to delivery cancer/tissue targeted theranostic agent for diagnostic and/or therapeutic applications. FIG. 2 is a flow chart of an exemplary diagnosing method according to an embodiment of the present invention. In one embodiment, a method 200 starts with preparing a subject to be diagnosed (block 202). Next, the front end 11a of the tubular body 11 of the endoscopic apparatus 1 is introduced into the large intestine of the subject to be diagnosed, so that the front end 11a reaches a position at a predetermined distance from a portion to be diagnosed (block 204). The predetermined distance is set for obtaining an optical diagnosing effect. After the front end 11a has reached the position at the predetermined distance from the portion to be diagnosed, the theranostic HEVNPs are supplied from the theranostic agent source 15 through the fluid channel 14 to the portion to be diagnosed, until the entire portion to be diagnosed is ready to be examined (block 206). Then, the portion to be diagnosed is examined using the imaging system 12, and the examination result is displayed on the image displaying system 13 to the operator (block 208). After the examination is finished, the endoscopic apparatus 1 is retracted from the intestine of the subject.

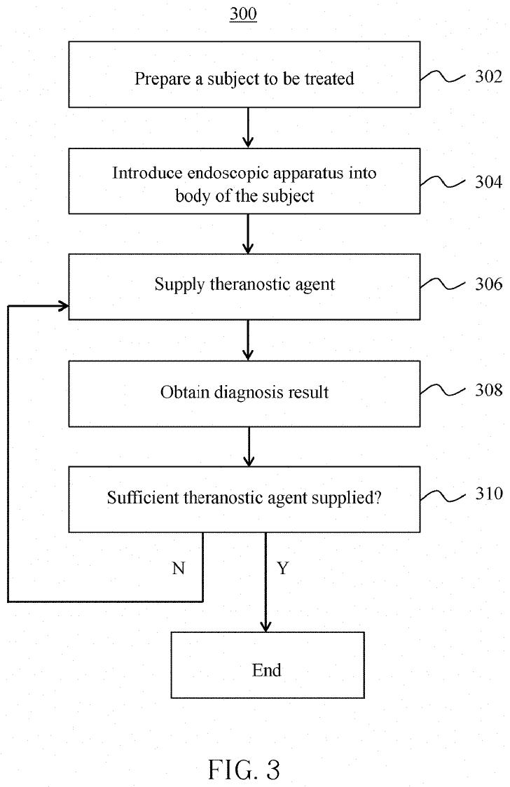

FIG. 3 is a flow chart of an exemplary treating method according to an embodiment of the present invention. In one embodiment, a method 300 starts with preparing a subject to be treated (block 302). Next, the front end 11a of the tubular body 11 of the endoscopic apparatus 1 is introduced into the body of the subject to be treated, so that the front end 11a reaches a position at a predetermined distance from a portion to be treated (block 304). The predetermined distance is set for obtaining an optical treating effect. After the front end 11a has reached the position at the predetermined distance from the portion to be treated, a predetermined amount of the theranostic agent is supplied from the theranostic agent source 15 through the fluid channel 14 to the portion to be treated (block 306). Then, the portion to be treated is examined using the imaging system 12, and the examination result is displayed on the image displaying system 13 to the operator for determining whether the supplied amount of theranostic agent is sufficient for therapy (block 308). In block 310, if the supplied amount of theranostic agent is determined to be insufficient, the flow goes back to block 306 for supplying an additional amount of theranostic agent, and then the operation in block 308 is performed again for examining the result of supplying. Such a cycle may be repeated one or more times to achieve a desired result of treatment. On the other hand, in block 310, once the supplied amount of theranostic agent is determined to be sufficient for therapy, the endoscopic apparatus 1 is retracted from the body of the subject to be treated.

During the introduction operations in blocks 204 and 304, the location of the front end 11a of the tubular body 11 may be monitored from images captured by the imaging system 12 and displayed by the image displaying system 13.

For the operations in blocks 208 and 308, the determination of the condition of the theranostic agent supplied on the portion to be diagnosed and/or treated may be facilitated by detecting fluorescence emitted from a fluorescent dye in the theranostic agent supplied to the portion to be diagnosed and/or treated.

The method described in this invention may be performed by health care providers using the endoscopic apparatus of the present invention as described above. For example, the method of the present invention may be applied to gastrointestinal tract (GI tract) endoscopy, including esophagogastroduodenoscopy (such as for esophagus, stomach, and duodenum), enteroscopy (such as for small intestines), colonoscopy/sigmoidoscopy (such as for large intestines/colons), magnification endoscopy, bile duct endoscopy, endoscopic retrograde cholangiopancreatography (ERCP), duodenoscope-assisted cholangiopancreatoscopy, intraoperative cholangioscopy, rectoscopy (for rectums), and anoscopy (for anuses). Rectoscopy and anoscopy can both be referred to as proctoscopy.

For example, the method of the present invention may be applied to respiratory tract endoscopy such rhinoscopy (for noses) and/or bronchoscopy (for lower respiratory tracts); otoscopy (for ears); cystoscopy (for urinary tracts); gynoscopy (for female reproductive systems) including colposcopy (for cervixes), hysteroscopy (for uteruses), and falloposcopy (for fallopian tubes).

As another example, the method of the present invention may be employed to diagnose and/or treat (through a small incision, for example) body cavities that are normally closed. Such cavities includes abdominal or pelvic cavities (laparoscopy), interior of joints (arthroscopy), and/or organs of chest (thoracoscopy and mediastinoscopy).

In some embodiments, the cancer/tissue targeted theranostic agent is a composition including a modified capsid protein. In some embodiments, the modified capsid protein is less than full length of HEV ORF2 protein; it comprises the M domain, segment 318-451 of the HEV ORF 2 protein of SEQ ID NO:1, 2, 3, 4, 5, or 6; and it comprises a heterologous polypeptide sequence inserted into the portion of HEV ORF2 protein within segment 342-344, 402-409 of SEQ ID NO:1, 2, 3, 4, 5, or 6. In some embodiments, the heterologous polypeptide sequence is inserted immediately after residue T342 or E407 of SEQ ID NO:1, 2, 3, 4, 5, or 6. In some embodiments, the heterologous polypeptide may be involved in targeting cancer cells for delivery of diagnostics and/or therapeutics, for example, the most widely used homing peptide, RGD (Arg-Gly-Asp) peptide or cyclic RGD peptide, which shows strong affinity for integrins vb 3 and vb 5, or homing peptides that specifically target HCC include TTPRDAY, FQHPSFI (HCBP1), SFSIIHTPILPL (SP94), RGWCRPLPKGEG (HCl), AGKGTPSLETTP (A54), KSLSRHDHIHHH (HCC79) and AWYPLPP.

In some embodiments, the modified capsid protein is able to form an acid and proteolytically stable HEV VLP and has at least one residue T342, or E407 of SEQ ID NO:1, 2, 3, 4, 5, or 6 substituted with a cysteine or lysine, and the cysteine or lysine is optionally chemically derivatized. In some embodiments, the cysteine or lysine is alkylated, acylated, arylated, succinylated, oxidized, or conjugated to a detectable label or cancer cell targeting ligand. For example, the detectable label may comprise a fluorophore, a superparamagnetic label, an MRI contrast agent, a positron emitting isotope, or a cluster of elements of group 3 through 18 having an atomic number greater than 20. In some embodiments, the detectable label comprises a gold nanocluster. In another example, the cancer cell targeting ligand is the heterologous polypeptide may be involved in targeting cancer cells for delivery of theranostic, for example, the most widely used homing peptide, RGD (Arg-Gly-Asp) or cyclic RGD peptide, or homing peptides that specifically target HCC include TTPRDAY, FQHPSFI (HCBP1), SFSIIHTPILPL (SP94), RGWCRPLPKGEG (HCl), AGKGTPSLETTP (AM), KSLSRHDHIHHH (HCC79) and AWYPLPP.

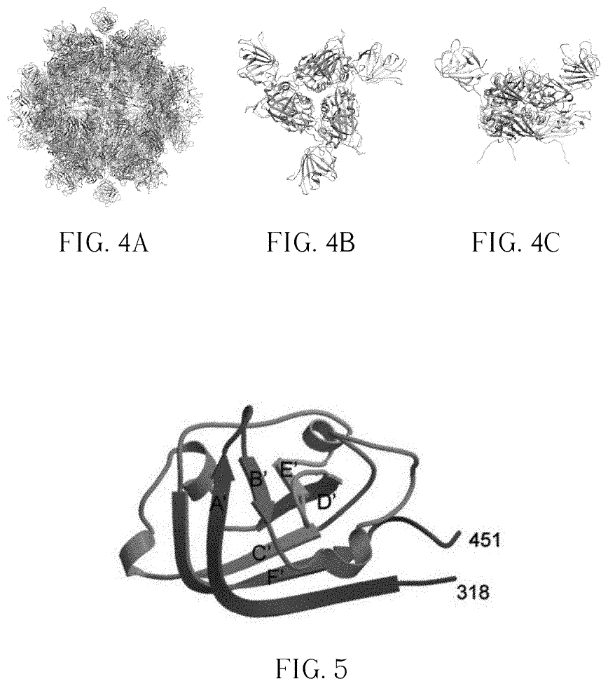

FIGS. 4A-4C illustrate structures of HEV-VLP presented as secondary structure for the M domain. FIG. 4A shows a PORF2 dimer viewed along the 2-fold axis of virus-like particle (VLP). FIG. 4B shows a top view of a 3-fold axis of HEV-VLP. The M domain is tightly associated with the S domain and located on the surface around the icosahedral 3-fold axis. FIG. 4C shows a side view of two M domains in HEV-VLP.

FIG. 5 illustrates the structure of M domain presented as a secondary structure. The M domain (amino acids 318-451) is one of the characteristic domains of HEVNP which has a twisted anti-parallel (3-barrel structure composed of 6 β-strands and 4 short α-helices.

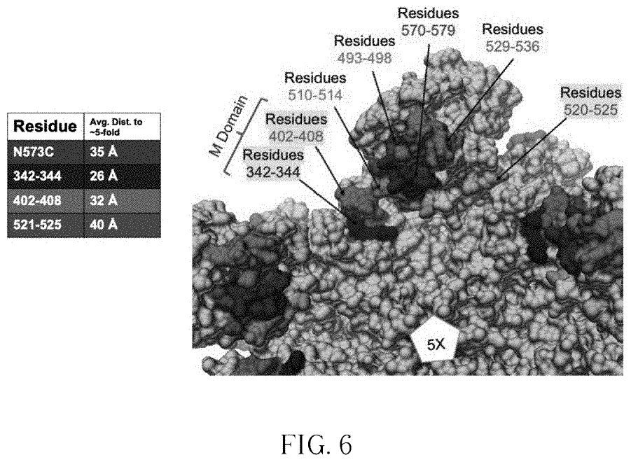

FIG. 6 illustrates structures of HEV-VLP and HEV PORF2 dimer presented as a solid surface. The distance between five-fold center of HEVNP and the aa N573C and various loops, 342-344, 402-408 on Middle (M) domain are estimated from the atomic structure.

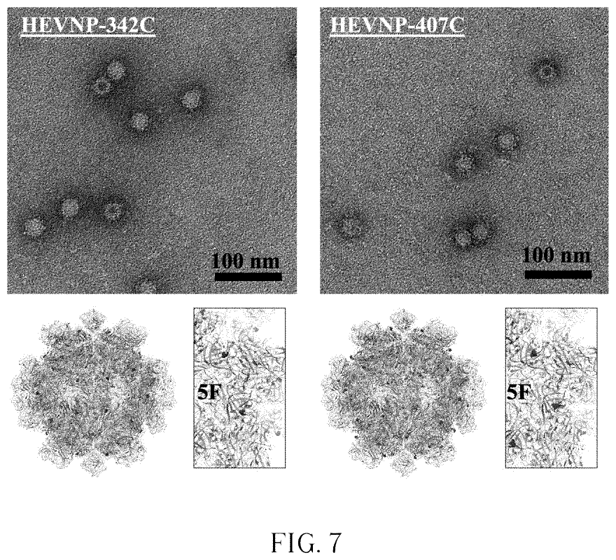

FIG. 7 illustrates similar VLP formation of all two HEV-Cys VLPs represented by electron micrographs of negative stained VLPs. In FIG. 7, purified HEV-342C VLPs are shown in the upper left part, and the 342C aa (small dark spots in the lower right part of FIG. 7) locates relatively to five-fold center of HEV VLP. Further, HEV-407C VLPs are shown in the upper right part of FIG. 7, and the 407C aa (small dark spots in the lower left part of FIG. 7) locates relatively to five-fold center of HEV VLP. In FIG. 7, bars represent 100 nm.

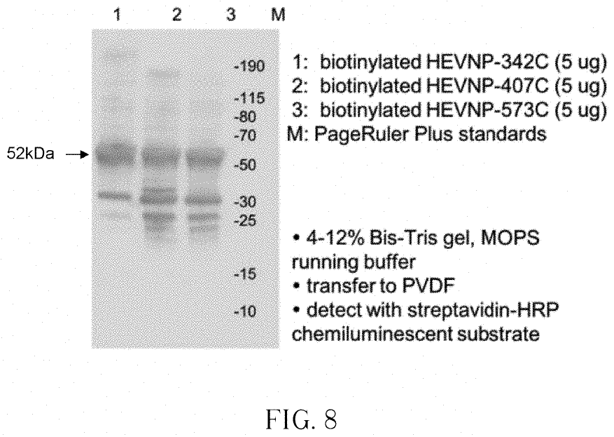

FIG. 8 illustrates surface exposure assay of Cys sites on P domains of HEV-573C, M domains of HEV-342C and HEV-407C. It was done by conjugating maleimide-biotin with HEV-Cys-VLPs at VLP conformation. The conjugated biotins were then detected by HRP conjugated Strapdevidin in SDS PAGE protein gel. The strongest signals showed at the size ˜52 kDa, which is the size of HEV capsid protein, ORF2. The bands at smaller sizes may correspond to the defected CPs.

The term “computer-readable media” is non-transitory computer-storage media. For example, non-transitory computer-storage media may include, but are not limited to, magnetic storage devices (e.g., hard disk, floppy disk, and magnetic strips), optical disks (e.g., compact disk (CD) and digital versatile disk (DVD)), smart cards, flash memory devices (e.g., thumb drive, stick, key drive, and SD cards), and volatile and non-volatile memory (e.g., random access memory (RAM), read-only memory (ROM)). Similarly, the term “machine-readable media” is non-transitory machine-storage media. Likewise, the term “processor-readable media” is non-transitory processor-storage media.

A non-transitory computer-readable storage medium can cause a machine to perform the functions or operations described, and includes any mechanism that stores information in a form accessible by a machine (e.g., computing device, electronic system, etc.), such as recordable/non-recordable media (e.g., read-only memory (ROM), random access memory (RAM), magnetic disk storage media, optical storage media, flash memory devices, etc.). A communication interface includes any mechanism that interfaces to any of a hardwired, wireless, optical, etc., medium to communicate to another device, such as a memory bus interface, a processor bus interface, an Internet connection, a disk controller, etc. The communication interface is configured by providing configuration parameters or sending signals to prepare the communication interface to provide a data signal describing the software content. The communication interface can be accessed via one or more commands or signals sent to the communication interface.

In some embodiments, a non-transitory computer readable medium is configured to store a program causing a computer, for example, to execute the method set forth in the present disclosure.

With the apparatus and the method provided in the present invention, it is possible to deliver theranostic agents more accurately to portions to be diagnosed and/or treated, compared to conventional ways for delivering theranostic agents. In addition, through visual confirmation provided by the apparatus and method of the present invention, it is also possible to precisely supply the theranostic agents to the portions to be diagnosed and/or treated, thereby reducing waste of theranostic agents.

Reference herein to “one embodiment” or “an embodiment” refers to one or more features, structures, materials, or characteristics described at least one example embodiment of the technology described herein. It does not denote or imply that the features, structures, materials, or characteristics are present in every embodiment. Thus, the appearances of the phrases “in one embodiment” or “in an embodiment” in various places throughout this document are not necessarily referring to the same embodiment of the technology. Furthermore, the features, structures, materials, or characteristics may be combined in any suitable manner in one or more embodiments.

In the above description of example implementations, for purposes of explanation, specific numbers, materials configurations, and other details are set forth to explain better the present invention, as claimed. However, it will be apparent to one skilled in the art that the claimed invention may be practiced using different details than the example ones described herein. In other instances, well-known features are omitted or simplified to clarify the description of the example implementations.

The inventors intend the described example implementations to be primarily examples. The inventors do not intend these example implementations to limit the scope of the appended claims. Rather, the inventors have contemplated that the claimed invention might also be embodied and implemented in other ways, in conjunction with other present or future technologies.

Moreover, the word “example” is used herein to mean serving as an example, instance, or illustration. Any aspect or design described herein as “example” is not necessarily to be construed as preferred or advantageous over other aspects or designs. Rather, use of the word example is intended to present concepts and techniques in a concrete fashion. The term “techniques,” for instance, may refer to one or more devices, apparatuses, systems, methods, articles of manufacture, and computer-readable instructions as indicated by the context described herein.

As used in this application, the term “or” is intended to mean an inclusive “or” rather than an exclusive “or.” That is unless specified otherwise or clear from context, “X employs A or B” is intended to mean any of the natural inclusive permutations. That is if X employs A; X employs B; or X employs both A and B, then “X employs A or B” is satisfied under any of the preceding instances. Also, the articles “an” and “an” as used in this application and the appended claims should be construed to mean “one or more,” unless specified otherwise or clear from context to be directed to a singular form.

These processes are illustrated as a collection of blocks in a logical flow graph, which represents a sequence of operations that can be implemented in mechanics alone or a combination of hardware, software, and firmware. In the context of software/firmware, the blocks represent instructions stored on one or more computer-readable storage media that, when executed by one or more processors, perform the recited operations.

Note that the order in which the processes are described is not intended to be construed as a limitation and any number of the described process blocks can be combined in any order to implement the processes or an alternate process. Additionally, individual blocks may be deleted from the processes without departing from the spirit and scope of the subject matter described herein.

In the claims appended herein, the inventors invoke 35 U.S.C. § 112(f) only when the words “means for” or “steps for” are used in the claim. If such words are not used in a claim, then the inventors do not intend for the claim to be construed to cover the corresponding structure, material, or acts described herein (and equivalents thereof) in accordance with 35 U.S.C. 112(f).

1. An endoscopic apparatus, comprising:
a tubular body having a front end and an operating end, the tubular body comprising a fluid channel disposed inside the tubular body, the fluid channel having a fluid inlet at the operating end and a fluid outlet at the front end;
a theranostic agent source coupled to the fluid inlet of the fluid channel and configured to supply a theranostic agent through the fluid channel; and
a controller coupled to the operating end of the tubular body, wherein the controller is configured to control the operations of the endoscopic apparatus.

2. The endoscopic apparatus of claim 1, further comprising an instrument channel through which a surgery tool reaches a portion to be diagnosed and treated for performing a surgery to the portion to be diagnosed and treated.

3. The endoscopic apparatus of claim 1, further comprising an image transmitting device disposed inside the tubular body, wherein the tubular body is flexible, and wherein the image transmitting device is an optical fiber device.

4. The endoscopic apparatus of claim 1, further comprising an image transmitting device disposed inside the tubular body, wherein the tubular body is rigid, and wherein the image transmitting device is a relay lens system.

5. The endoscopic apparatus of claim 1, further comprising an displaying system coupled to the operating end of the tubular body, wherein the image displaying system is an eyepiece or a camera.

6. A method of delivering a theranostic agent using an endoscopic apparatus comprising a tubular body, an imaging system disposed inside the tubular body, an image displaying system coupled to the imaging system, an illumination system disposed inside the tubular body, a fluid channel disposed inside the tubular body, and a theranostic agent source coupled to the fluid channel, the method comprising:
introducing the endoscopic apparatus into a body of a subject having a portion to be diagnosed and/or treated;
illuminating an area around a front end of the endoscopic apparatus introduced into the body of the subject;
delivering the theranostic agent from the theranostic agent source through the fluid channel to a portion to be diagnosed and/or treated;
capturing, using the imaging system, images within the area around the front end of the endoscopic apparatus;
displaying images captured by the imaging system with the image displaying system; and
determining the condition of the theranostic agent supplied on the portion to be diagnosed and treated based on the images captured by the imaging system and displayed on the image displaying system.

7. The method of claim 6, wherein the endoscopic apparatus comprises an instrument channel, and wherein the method further comprises performing a surgery to the portion to be diagnosed and treated with a surgery tool inserted through the instrument channel and reaching the portion to be diagnosed and treated.

8. The method of claim 6, further comprising during the introduction, monitoring the location of a front end of the tubular body from images captured by the imaging system and displayed by the image displaying system.

9. The method of claim 6, wherein the determining the condition of the theranostic agent supplied on the portion to be diagnosed and treated comprises detecting fluorescence emitted from a fluorescent dye in the theranostic agent supplied to the portion to be diagnosed and treated.

10. A non-transitory computer readable medium storing a program causing a computer to execute the method of claim 6.

11. A composition delivered using the method of claim 6, comprising:
a modified capsid protein that comprises at least a portion of hepatitis E virus (HEV) open Reading Frame 2 (ORF2) protein and is able to form an HEV virus like particle (VLP).

12. The composition of claim 11, wherein the modified capsid protein is less than full length of HEV ORF2 protein, comprises the M domain, segment 318-451, and/or the P domain, segment 452-606 of the HEV ORF 2 protein of SEQ ID NO:1, 2, 3, 4, 5, or 6, and comprises a heterologous polypeptide sequence inserted into the portion of HEV ORF2 protein within segment 342-344, 402-409, 483-490, 530-535, 554-561, 573-577, 582-593, or 601-603 of SEQ ID NO:1, 2, 3, 4, 5, or 6.

13. The composition of claim 12, wherein the heterologous polypeptide sequence is inserted immediately after residue T342, E407, Y485 of SEQ ID NO:1, 2, 3, 4, 5, or 6.

14. The composition of claim 12, wherein the heterologous polypeptide is a RGD or cyclic RGD peptide.

15. The composition of claim 11, wherein the modified capsid protein is able to form an acid and proteolytically stable HEV VLP and has at least one residue T342, E407, Y485, T489, 5533, N573, or T586 of SEQ ID NO:1, 2, 3, 4, 5, or 6 substituted with a cysteine or lysine, which is optionally chemically derivatized.

16. The composition of claim 14, wherein the cysteine or lysine is alkylated, acylated, arylated, succinylated, oxidized, or conjugated to a detectable label or liver cell targeting ligand.

17. The composition of claim 16, wherein the detectable label comprises a fluorophore, a superparamagnetic label, an MRI contrast agent, a positron emitting isotope, or a cluster of elements of group 3 through 18 having an atomic number greater than 20.

18. The composition of claim 17, wherein the detectable label comprises a gold nanocluster, and/or fluorescence dye.

19. The composition of claim 16, wherein the cancer cell targeting ligand is a RGD or cyclic RGD peptide.

20. The composition of claim 19, further comprising a pharmaceutically acceptable excipient.


SEQUENCE LISTING (11 sequences)

SEQ ID NO: 1 - protein, 660 aa, Hepatitis E virus
  MISC_FEATURE: capsid protein genotype 1 (NP_056788.1), residues 1-660
MRPRPILLLLLMFLPMLPAPPPGQPSGRRRGRRSGGSGGGFWGDRADSQPFAIPYIHPTNPFAPDVTAAAGAGPRVRQPARPLGSAWRDQAQRPAAASRRRPTTAGAAPLTAVAPAHDTPPVPDVDSRGAILRRQYNLSTSPLTSSVATGTNLVLYAAPLSPLLPLQDGTNTHIMATEASNYAQYRVVRATIRYRPLVPNAVGGYAISISFWPQTTTTPTSVDMNSITSTDVRILVQPGIASEHVIPSERLHYRNQGWRSVETSGVAEEEATSGLVMLCIHGSLVNSYTNTPYTGALGLLDFALELEFRNLTPGNTNTRVSRYSSTARHRLRRGADGTAELTTTAATRFMKDLYFTSTNGVGEIGRGIALTLFNLADTLLGGLPTELISSAGGQLFYSRPVVSANGEPTVKLYTSVENAQQDKGIAIPHDIDLGESRVVIQDYDNQHEQDRPTPSPAPSRPFSVLRANDVLWLSLTAAEYDQSTYGSSTGPVYVSDSVTLVNVATGAQAVARSLDWTKVTLDGRPLSTTQQYSKTFFVLPLRGKLSFWEAGTTKAGYPYNYNTTASDQLLVENAAGHRVAISTYTTSLGAGPVSISAVAVLAPHSALALLEDTMDYPARAHTFDDFCPECRPLGLQGCAFQSTVAELQRLKMKVGKTREL

SEQ ID NO: 2 - protein, 660 aa, Hepatitis E virus
  MISC_FEATURE: capsid protein genotype 3 (BAH10873.1), residues 1-660
MRPRAVLLLFFVLLPMLPAPPAGQPSGRRRGRRSGGAGGGFWGDRVDSQPFALPYIHPTNPFAADVVSQSGAGARPRQPPRPLGSAWRDQSQRPSAAPRRRSAPAGAAPLTAISPAPDTAPVPDVDSRGAILRRQYNLSTSPLTSSVASGTNLVLYAAPLNPLLPLQDGTNTHIMATEASNYAQYRVVRATIRYRPLVPNAVGGYAISISFWPQTTTTPTSVDMNSITSTDVRILVQPGIASELVIPSERLHYRNQGWRSVETTGVAEEEATSGLVMLCIHGSPVNSYTNTPYTGALGLLDFALELEFRNLTPGNTNTRVSRYTSTARHRLRRGADGTAELTTTAATRFMKDLHFTGTNGVGEVGRGIALTLFNLADTLLGGLPTELISSAGGQLFYSRPVVSANGEPTVKLYTSVENAQQDKGITIPHDIDLGDSRVVIQDYDNQHEQDRPTPSPAPSRPFSVLRANDVLWLSLTAAEYDQTTYGSSTNPMYVSDTVTFVNVATGAQAVARSLDWSKVTLDGRPLTTIQQYSKTFYVLPLRGKLSFWEAGTTKAGYPYNYNTTASDQILIENAAGHRVAISTYTTSLGAGPTSISAVGVLAPHSALAVLEDTTDYPARAHTFDDFCPECRTLGLQGCAFQSTIAELQRLKMKVGKTRES

SEQ ID NO: 3 - protein, 660 aa, Hepatitis E virus
  MISC_FEATURE: capsid protein genotype 4 (ABE27148.1), residues 1-660
MRPRAVLLLFFVLLPMLPAPPAGQPSGRRRGRRSGGTGGGFWGDRVDSQPFALPYIHPTNPFASDIPTATGAGARPRQPARPLGSAWRDQSQRPAAPARRRSAPAGASPLTAVAPAPDTAPVPDVDSRGAILRRQYNLSTSPLTSTIATGTNLVLYAAPLSPLLPLQDGTNTHIIATEASNYAQYRVVRATIRYRPLVPNAVGGYAISISFWPQTTTTPTSVDMNSITSTDVRILVQPGIASELVIPSERLHYRNQGWRSVETSGVAEEEATSGLVMLCIHGSPVNSYTNTPYTGALGLLDFALELEFRNLTPGNTNTRVSRYSSSARHKLCRGPDGTAELTTTAATRFMKDLHFTGTNGVGEVGRGIALTLLNLADTLLGGLPTELISSAGGQLFYSRPVVSANGEPTVKLYTSVENAQQDKGIAIPHDIDLGESRVVIQDYDNQHEQDRPTPSPAPSRPFSVLRANDVLWLSLTAAEYDQTTYGSSTNPMYVSDTVTFVNVATGTQGVSRSLDWSKVTLDGRPLTTIQQYSKTFFVLPLRGKLSFWEAGTTKAGYPYNYNTTASDQILIENAPGHRVCISTYTTNLGSGPVSISAVGVLAPHSALAALEDTVDYPARAHTFDDFCPECRALGLQGCAFQSTVAELQRLKMKVGKTQEY

SEQ ID NO: 4 - protein, 659 aa, Hepatitis E virus
  MISC_FEATURE: HEV capsid protein genotype 2 (M74506.1), residues 1-659
MRPRPLLLLFLLFLPMLPAPPTGQPSGRRRGRRSGGTGGGFWGDRVDSQPFAIPYIHPTNPFAPDVAAASGSGPRLRQPARPLGSTWRDQAQRPSAASRRRPATAGAAALTAVAPAHDTSPVPDVDSRGAILRRQYNLSTSPLTSSVASGTNLVLYAAPLNPPLPLQDGTNTHIMATEASNYAQYRVARATIRYRPLVPNAVGGYAISISFWPQTTTTPTSVDMNSITSTDVRILVQPGIASELVIPSERLHYRNQGWRSVETSGVAEEEATSGLVMLCIHGSPVNSYTNTPYTGALGLLDFALELEFRNLTTCNTNTRVSRYSSTARHSARGADGTAELTTTAATRFMKDLHFTGLNGVGEVGRGIALTLLNLADTLLGGLPTELISSAGGQLFYSRPVVSANGEPTVKLYTSVENAQQDKGVAIPHDIDLGDSRVVIQDYDNQHEQDRPTPSPAPSRPFSVLRANDVLWLSLTAAEYDQSTYGSSTGPVYISDSVTLVNVATGAQAVARSLDWSKVTLDGRPLPTVEQYSKTFFVLPLRGKLSFWEAGTTKAGYPYNYNTTASDQILIENAAGHRVAISTYTTRLGAGPVAISAAAVLAPRSALALLEDTFDYPGRAHTFDDFCPECRALGLQGCAFQSTVAELQRLKVKVGKTREL

SEQ ID NO: 5 - protein, 610 aa, Hepatitis E virus
  MISC_FEATURE: capsid protein genotype 5 (BAJ77116), residues 1-660
  misc_feature (368)..(368): Xaa can be any naturally occurring amino acid
MNNMFLCFACGYATMRPRAILLLLVVLLPMLPAPPAGQSSGRRRGRRSGGAGSGFWGDRVDSQPFALPYIHPTNPFASDTIAATGTGARSRQSARPLGSAWRDQTQRPPAASRRRSTPTGASPLTAVAPAPDTRPVPDVDSRGAILRRQYNLSTSPLTSTIASGTNLVLYAAPLSPLLPLQDGTNTHIMATEASNYAQYRVVRATIRYRPLVPNAVGGYAISISFWPQTTTTPTSVDMNSITSTDVRIVVQPGLASELVIPSERLHYRNQGWRSVETSGVAEEEATSGLVMLCIHGSPVNSYTNTPYTGALGLLDFALELEFRNLTPGNTNTRVSRYSSTARHRLHRGADGTAELTTTAATRFMKDLXFTGSNGIGEVGRGIALTLFNLADTLLGGLPTELISSAGGQLFYSRPVVSANGEPTVKLYTSVENAQQDKGIAIPHDIDLGDSRVVIQDYDNQHEQDRPTPSPAPSRPFSVLRVNDVLWLTMTAAEYDQTTYGTSTDPVYVSDTVTFVNVATGAQGVARSLDWSKVTLDGRPLTTIQRHSKNYFVLPLRGKLSFWEAGTTKAGYPYNYNTTASDQILIENAAGHRVCISTYTTSLGSGPVSVS

SEQ ID NO: 6 - protein, 610 aa, Hepatitis E virus
  MISC_FEATURE: capsid protein genotype 6 (BAJ61827.1), residues 1-610
MRPRAVLLLFLMLLPMLPAPPAGQPSGRRRGRRSGGSGGGFWGDRVDSQPFALPYIHPTNPFASDVSTSAGAGARARQAARPLGSAWRDQSQRPSASARRRPTPAGASPLTAVAPAPDTTPVPDVDSRGAILRRQYNLSTSPLTSTVASGTNLVLYAAPLGPLLPLQDGTNTHIMATEASNYAQYRVIRATIRYRPLVPNAVGGYAISISFWPQTTTTPTSVDMNSITSTDVRILVQPGLASELIIPSERLHYRNQGWRSVETSGVAEEEATSGLVMLCIHGSPVNSYTNTPYTGALGLLDFALELEFRNLTPGNTNTRVSRYTSTARHRLRRGPDGTAELTTTAATRFMKDLYFTGSNGLGEVGRGIALTLFNLADTLLGGLPTELISSAGGQLFYSRPVVSANGEPTVKLYTSVENAQQDKGIAIPHEIDLGDSRVTIQDYDNQHEQDRPTPSPAPSRPFSVLRVNDVLWLTLTAAEYDQTTYGSTTNPMYVSDTVTFVNVATGAQGVARALDWSKVTFDGRPLTTVQQYGKSFFVLPLRGKLSFWEAGTVKAGYPYNYNTTASDQILVENAPGHRVCISTYTTNLGSGPVSISAVGVLAPHAATAAL

SEQ ID NO: 7 - protein, 134 aa, Hepatitis E virus
  MISC_FEATURE: genotype 1 (NP_056788.1), The M domain, residues 318-451
TRVSRYSSTARHRLRRGADGTAELTTTAATRFMKDLYFTSTNGVGEIGRGIALTLFNLADTLLGGLPTELISSAGGQLFYSRPVVSANGEPTVKLYTSVENAQQDKGIAIPHDIDLGESRVVIQDYDNQHEQDR

SEQ ID NO: 8 - protein, 134 aa, Hepatitis E virus
  MISC_FEATURE: genotype 3 (BAH10873.1), The M domain, residues 318-451
TRVSRYTSTARHRLRRGADGTAELTTTAATRFMKDLHFTGTNGVGEVGRGIALTLFNLADTLLGGLPTELISSAGGQLFYSRPVVSANGEPTVKLYTSVENAQQDKGITIPHDIDLGDSRVVIQDYDNQHEQDR

SEQ ID NO: 9 - protein, 134 aa, Hepatitis E virus
  MISC_FEATURE: genotype 4 (ABE27148.1), The M domain, residues 318-451
TRVSRYSSSARHKLCRGPDGTAELTTTAATRFMKDLHFTGTNGVGEVGRGIALTLLNLADTLLGGLPTELISSAGGQLFYSRPVVSANGEPTVKLYTSVENAQQDKGIAIPHDIDLGESRVVIQDYDNQHEQDR

SEQ ID NO: 10 - protein, 134 aa, Hepatitis E virus
  MISC_FEATURE: genotype 2 (M74506.1), The M domain. residues 318-451
TRVSRYSSTARHSARGADGTAELTTTAATRFMKDLHFTGLNGVGEVGRGIALTLLNLADTLLGGLPTELISSAGGQLFYSRPVVSANGEPTVKLYTSVENAQQDKGVAIPHDIDLGDSRVVIQDYDNQHEQDRP

SEQ ID NO: 11 - protein, 134 aa, Hepatitis E virus
  MISC_FEATURE: genotype 5(BAJ77116), The M domain. residues 318-451
  misc_feature (51)..(51): Xaa can be any naturally occurring amino acid
LELEFRNLTPGNTNTRVSRYSSTARHRLHRGADGTAELTTTAATRFMKDLXFTGSNGIGEVGRGIALTLFNLADTLLGGLPTELISSAGGQLFYSRPVVSANGEPTVKLYTSVENAQQDKGIAIPHDIDLGDSR